(19) United States
(12) Patent Application Publication
(10) Pub. No.: US 2019/0006027 A1
(43) Pub. Date: Jan. 3, 2019

(54) AUTOMATIC IDENTIFICATION AND EXTRACTION OF MEDICAL CONDITIONS AND EVIDENCES FROM ELECTRONIC HEALTH RECORDS

(71) Applicant: Accenture Global Solutions Limited, Dublin (IE)

(72) Inventors: Bogdan E. Sacaleanu, Dublin (IE); Pedro Sacristan, Dublin (IE); Urvesh Bhowan, Co. Wicklow (IE); Medb Corcoran, Dublin (IE); Jivan Virdee, London (GB); James Robert Priestas, Arlington, VA (US); Tara Lynn O'Gara, Arlington, VA (US); Thomas D. Perry, Vienna, VA (US); Theresa M. Gaffney, Milton, MA (US); Meghan Hildebrand Fotopoulos, Philadelphia, PA (US); Laura O'Malley, Dublin (IE)

(21) Appl. No.: 15/879,031

(22) Filed: Jan. 24, 2018

Related U.S. Application Data
(60) Provisional application No. 62/527,441, filed on Jun. 30, 2017.

(51) Int. Cl. G16H 10/60; G16H 50/20; G06F 17/30

(52) U.S. Cl. CPC G16H 10/60; G16H 50/30; G06F 17/30477; G16H 50/20

(57) ABSTRACT

This document describes systems, methods, devices, and other techniques for automatically identifying and extracting medical conditions and supporting evidences from electronic health records. In some implementations, formatted text extracted from an unstructured electronic health record is obtained. The formatted text is segmented into multiple documents, wherein each document comprises a respective document type and represents a respective document encounter. Medical condition entities and supporting evidence entities referenced in each of the multiple documents are extracted. Extracted supporting evidence entities within a same document are linked to respective extracted medical condition entities from the same document using one or more of i) medical ontologies, or ii) a medical knowledge base. Output data representing linked supporting evidence entities and medical condition entities within a same document is provided.

### CROSS-REFERENCE TO RELATED APPLICATIONS

This application claims the benefit of U.S. Prov. App. No. 62/527,441, filed on Jun. 30, 2017, the disclosure of which is expressly incorporated herein by reference in the entirety.

### TECHNICAL FIELD

This specification generally describes methods and systems for processing data representing electronic health records.

### BACKGROUND

Healthcare providers and health insurers are often required to manually review unstructured patient electronic health records to identify patient medical conditions and supporting evidences. Such medical conditions and supporting evidences may be used to diagnose diseases or conditions that explain a patients symptoms, or to claim health insurance reimbursements.

Manual review of electronic health records is a complex task. In addition, manual reviews may be time consuming, and error prone since medical conditions and supporting evidences can potentially be missed. Electronic health records typically represent a patient's medical history over an extended period of time, and include a collection of clinical notes from different physicians per consultation, prescriptions, hospital admission or discharge forms, laboratory order forms and results, clinical review transactions, letters of referral, or procedure notes. Automating the review of electronic health records is also complex due to the heterogeneity of electronic health record documents.

### SUMMARY

This specification describes systems and methods for automatic identification and extraction of medical conditions and evidences supporting those conditions such as medications, symptoms, treatments, or laboratory results in electronic patient medical records.

Innovative aspects of the subject matter described in this specification may be embodied in methods for automatically identifying and extracting medical conditions and supporting evidences from electronic health records, the methods including the actions of obtaining formatted text extracted from an unstructured electronic health record; segmenting the formatted text into multiple documents, each document comprising a respective document type and represents a respective document encounter; extracting, from each document, one or more entities referenced in the document, the entities comprising medical condition entities and supporting evidence entities; linking, within each document, one or more of the extracted supporting evidence entities to respective extracted medical condition entities using one or more of i) medical ontologies, or ii) a medical knowledge base; and providing, for each document, output data representing linked supporting evidence entities and medical condition entities.

Other embodiments of this aspect include corresponding computer systems, apparatus, and computer programs recorded on one or more computer storage devices, each configured to perform the actions of the methods. A system of one or more computers can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus (e.g., one or more computers or computer processors), cause the apparatus to perform the actions.

The foregoing and other embodiments can each optionally include one or more of the following features, alone or in combination. In some implementations segmenting the formatted text into multiple documents comprises: analyzing the formatted text to calculate multiple feature vectors of numerical features that characterize respective portions of the formatted text; providing the calculated feature vectors as inputs to a first classifier, wherein the first classifier is configured to predict whether a portion of text represents a document boundary or not; and segmenting the formatted text into multiple documents by creating document boundaries between portions of text based on outputs received from the first classifier.

In some implementations the method further comprises providing the calculated feature vectors as inputs to a second classifier, wherein the second classifier is configured to predict whether a portion of text is relevant or not; and removing irrelevant portions of text from the formatted text based on outputs received from the second classifier.

In some implementations the numerical features comprise one or more of lexical features, language features or entity features.

In some implementations evidence entities comprise entities of respective semantic types, the semantic types comprising one or more of i) medications, ii) symptoms, iii) laboratory results, iv) tests ordered, v) treatments, vi) assessments, or vii) historic medical conditions.

In some implementations extracting, from each document, one or more entities referenced in the document, wherein the entities comprise condition entities and supporting evidence entities comprises: applying one or more of i) natural language processing techniques, ii) entity extraction techniques, or iii) medical ontologies to identify one or more medical condition entities and evidence entities in each document; and identifying and removing irrelevant entities, comprising applying domain specific indicators including one or more of i) lexical terms, ii) short terms, iii) context terms, iv) entities mentioned in reference.

In some implementations the method further comprises categorizing the identified evidence entities by semantic entity type, and wherein the provided data representing linked medical condition entities and supporting evidence entities comprises data indicating which categories the linked medical condition entities and supporting evidence entities belong to.

In some implementations linking, within each document, one or more of the extracted supporting evidence entities to respective extracted medical condition entities using one or more of i) medical ontologies, or ii) a medical knowledge base comprises: accessing medical ontologies to identify a set of candidate relations between the extracted medical condition entities and any evidence entities that occur in the same document; querying a knowledge base to determine whether any of the relations in the identified set of relations are invalid; in response to determining that one or more of the relations are invalid, removing the invalid relations from the identified set of relations; querying the knowledge base to identify new relations between the extracted medical condition entities and any evidence entities that occur in the same document.

In some implementations providing, for each document, output data representing linked supporting evidence entities and medical condition entities comprises: assigning the identified medical condition entities a relevance score based on features of the medical condition, wherein features of the medical condition comprise one or more of i) context within the document, or ii) quality of supporting evidences linked to the medical condition; ranking the scored medical condition entities to determine a representative subset of condition entities of predetermined size; assigning the identified supporting evidence entities respective relevance scores based on features of the evidence entities; providing, as output, data representing linked supporting evidence entities and medical condition entities whose relevance scores exceed a predetermined threshold.

In some implementations providing, for each document, output data representing linked supporting evidence entities and medical condition entities comprises providing data representing an interactive graphical user interface that visualizes document boundaries and the linked supporting evidences and medical condition entities as annotations over a plain text representation of the electronic health record.

In some implementations providing data representing an interactive graphical user interface that visualizes the linked supporting evidences and medical condition entities as annotations over a plain text representation of the electronic health record comprises: converting data representing the electronic health record into a Hypertext Markup Language format; parsing the converted data to extract electronic health record styling information, wherein styling information comprises one or more of i) text headings, ii) text typeface, iii) text colours, iv) structure of text; and using the extracted styling information to generate the interactive graphical user interface.

In some implementations providing, for each document, output data representing linked supporting evidence entities and medical condition entities comprises providing data representing an interactive graphical user interface that visualizes document boundaries and a predetermined number of relevant linked supporting evidences and medical condition entities as annotations over a plain text representation of the electronic health record.

In some implementations the plain text representation of the electronic health record comprises relevant portions of text extracted from the electronic health record.

In some implementations the method further comprises receiving user input through the interactive graphical user interface, the user input indicating edits to one or more of i) the visualized document boundaries or ii) the linked supporting evidences and medical condition entities; and updating the knowledge base based on the edits indicated by the received user input.

In some implementations the method further comprises converting unstructured data in the unstructured electronic health record to the formatted text.

In some implementations obtaining formatted text extracted from an unstructured electronic health record comprises: receiving input data representing the unstructured electronic health record; converting the received input data into a Hypertext Markup Language format; and extracting formatted text by parsing the Hypertext Markup Language.

Some implementations of the subject matter described herein may realize, in certain instances, one or more of the following advantages. In some implementations, a system implementing techniques for automatic identification and extraction of medical conditions and evidences from electronic health records, as described in this specification, may be used to review medical records and increase throughput, e.g., volume of processed patient charts, compared to other systems that do not implement the techniques described herein. This may result in improved healthcare services provided to patients, since patients may be diagnosed or treated more quickly. In addition, a system implementing techniques for automatic identification and extraction of medical conditions and evidences from electronic health records, as described in this specification, may achieve an increase in accuracy of identified medical conditions and supporting evidences compared to other systems that do not implement the techniques described herein. Increased accuracy of identified medical conditions may result in improved healthcare services provided to patients.

The details of one or more embodiments of the subject matter described in this specification are set forth in the accompanying drawings and the description below. Other potential features, aspects, and advantages of the subject matter will become apparent from the description, the drawings, and the claims.

### DESCRIPTION OF DRAWINGS

FIG. 1 illustrates an example process for automatically identifying and extracting medical conditions and evidences from an electronic health record.

FIG. 2 is a block diagram of an example system for automatically identifying and extracting medical conditions and evidences from an electronic health record.

FIG. 3 is a flowchart of an example process for generating linked medical condition entities and supporting evidence entities from an electronic health record.

FIG. 4 is a flowchart of an example process for segmenting formatted text extracted from an electronic health record into multiple portions of text.

FIG. 5 is a flowchart of an example process for linking extracted medical condition entities to supporting evidence entities.

FIG. 6 is a flowchart of an example process for scoring linked medical condition entities and supporting evidence entities.

FIG. 7 is an illustration of an example graphical user interface.

FIG. 8 illustrates a schematic diagram of an example computer system.

Like reference symbols in the various drawings indicate like elements.

### DETAILED DESCRIPTION

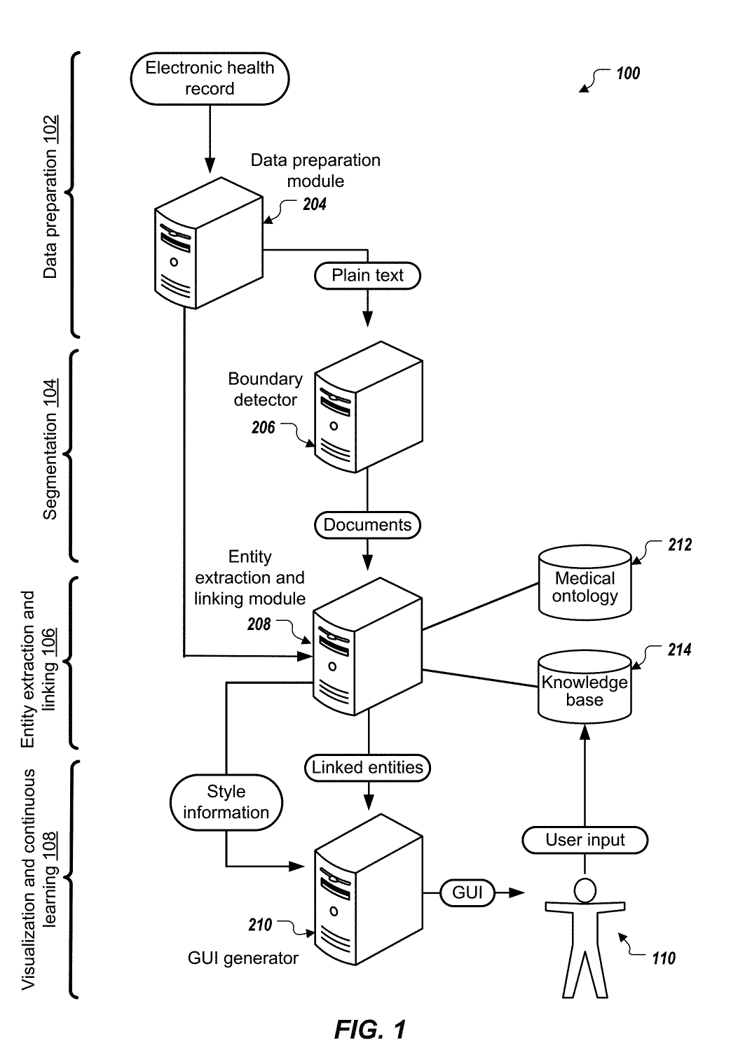

FIG. 1 is a block diagram 100 of an example computing system performing an example process for identifying and extracting medical conditions and evidences from an electronic health record. For convenience, the block diagram 100 illustrates the example process as including four stages—a data preparation stage 102, a segmentation stage 104, an entity extraction and linking stage 106, and a visualization and continuous learning stage 108. However, in some implementations the example process may include fewer or more stages. For convenience, each of the four stages are illustrated as being performed by respective modules of the computing system, e.g., a data preparation module 204, boundary detection module 206, entity extraction and linking module 208, and a graphical user interface (GUI) generator 210. However, in some implementations stages of a process for identifying and extracting medical conditions and evidences from an electronic health record may be performed by other computing modules.

During the data preparation stage 102, the data preparation module 204 receives data representing an unstructured electronic health record (EHR), e.g., data representing a PDF version of the electronic health record. An EHR is a systematic collection of a patient's health information stored in a digital format. For example, the EHR may include data representing a patient's medical history, including but not limited to data representing physician assessments, prescribed medications, allergies, immunization status, received laboratory test results, radiology images, vital sign statistics, personal statistics such as weight and height, and billing information. An EHR captures the state of a patient's health over time in a single modifiable file that is shared across different health care providers and services. The data preparation module 204 extracts formatted text from the EHR and provides the formatted text to the boundary detection module 206.

During the segmentation stage 104, the boundary detection module 206 receives formatted text extracted from the EHR and segments the formatted text into multiple documents, each document including a portion of the text extracted from the EHR. The boundary detection module 206 segments the received formatted text into multiple documents based on document type. For example, the data preparation module may separate the received formatted text into respective documents representing physician notes, prescriptions, laboratory results, admission or discharge notes, letters of referral, procedure notes or radiology images using machine learning techniques and/or business rules that detect boundaries between different encounters in the received data.

Segmenting the received formatted text into multiple documents in this manner provides improved context for the entity extraction stage 106 described below. For example, segmenting the received formatted text into multiple documents provides improved textual context for identifying, disambiguating and linking entities that appear in the individual documents, since semantics around an entity may be different depending on the document type. As another example, by only considering supporting evidence entities within individual documents, the scope of condition-to-evidence linking is reduced to entities that share a same context.

During the entity extraction and linking stage 106 the entity extraction and linking module 208 automatically identifies and extracts entities and relations between entities within the text of each of the multiple documents. In this context, entities include occurrences of medical conditions and supporting evidences, e.g., medications, symptoms, or treatments. To identify and extract entities from the text of each of the multiple documents the entity extraction and linking module 208 may apply natural language processing techniques. The entity extraction and linking module 208 may then apply reasoning techniques over multiple knowledge sources, e.g., including medical ontologies 212 and knowledge graphs or databases 214 to infer condition-evidence linking. The entity extraction and linking module 208 may further score and rank the extracted entities and condition-evidence links to generate a most-representative set of entities and condition-evidence links.

During the visualization and continuous learning stage 108 the GUI generator 210 processes data representing the most representative set of entities and condition-evidence links to generate a GUI that displays the extracted entities and entity relations in the set as annotations over a plain text representation of the EHR. In some implementations styling information, e.g., headings or text typeface, extracted from the EHR may be used to preserve the visual structure of the original EHR in the GUI, since styling information is often lost when extracting formatted text from a PDF document, e.g., using OCR techniques. For example, the system may provide styling information in the form of a separate mark-up over the plain text representation.

Treating the annotations, styling information and extracted text as separate items in the generated GUI allows for user interactions 110 with the system, e.g., edits, to be captured as feedback for continuous learning. For example, the GUI may be configured to receive user input that provides feedback relating to the generated annotations to improve the knowledge bases over time. User input such as validating or invalidating the extracted entities and entity relations may be modelled and captured in the knowledge base, and used to inform future decisions made by the system. In some cases the GUI display may facilitate the capture of these user interactions, and the styling information may make the EHR visually easier to manually review.

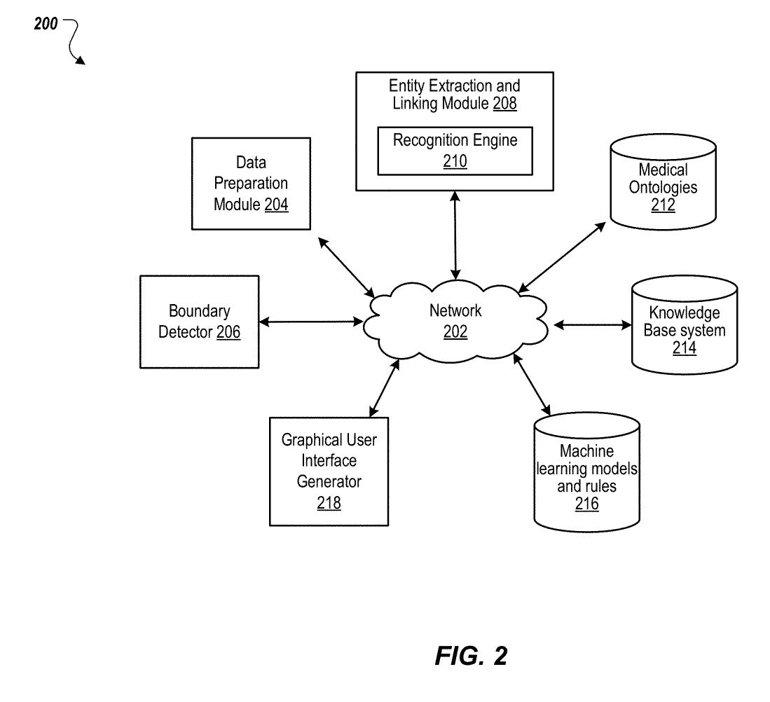

FIG. 2 is a block diagram of an example system 200 for automatically identifying and extracting medical conditions and evidences from an electronic health record. In some implementations, a computer network 202, such as a local area network (LAN), wide area network (WAN), the Internet, or a combination thereof, connects data preparation module 204, boundary detector 206, entity extraction and linking module 208, graphical user interface generator 218, machine learning models and rules database 216, knowledge base system 214 and medical ontologies 212. In some implementations, all or some of the data preparation module 204, boundary detector 206, entity extraction and linking module 208, graphical user interface generator 218, machine learning models and rules database 216, knowledge base system 214 and medical ontologies 212 can be implemented in a single computing system, and may communicate with none, one, or more other components over a network.

The data preparation module 204 is configured to extract text from an unstructured electronic health record. For example, the data preparation module 204 may be configured to receive data representing an electronic health record, e.g., a PDF file. The data preparation module 204 may include one or more data processing engines, e.g., an optical character recognition (OCR) engine, that are configured to convert the received data into machine encoded text, e.g., in Hypertext Markup Language (HTML) format. The data preparation module 204 may parse the machine encoded text to extract a formatted text representation of the electronic health record. The data preparation module 204 may provide the formatted text representation of the electronic health record to the boundary detection module 206.

In some implementations, the data preparation module 204 may be further configured to extract styling information from machine encoded text. For example, the data preparation module 204 may extract information that indicates whether a portion of the machine encoded text represents a text heading, was originally displayed as bold, underlined or italic font, was displayed in a particular colour, included a bulleted list, etc. The data preparation module 204 may provide the extracted styling information to the graphical user interface generator 210, as described in more detail below.

The boundary detection module 206 is configured to receive a formatted text representation of an electronic health record and to segment the received formatted text into multiple documents of different types, e.g., physician notes, laboratory results, or prescriptions, with each document representing a respective encounter, e.g., different physician appointments on different days or at different times, or prescriptions issued by different doctors and/or on different days or at different times.

To segment the received formatted text into multiple documents, the boundary detection module 206 generates feature vectors of numerical features that characterize respective portions of the formatted text, e.g., a set of feature vectors for each page of the formatted text. Example numerical features include one or more of lexical features, language features or entity features. Example lexical features include a number of lines, words, nouns or verbs in a portion of formatted text. Example language features include a percentage of words in a domain language such as English, or a number of different languages detected in a portion of text. Example entity features include a number of clinical terms such as diseases, medications, symptoms, tests, names or dates in a portion of text.

The boundary detection module 206 uses the generated feature vectors to segment the formatted text representing the electronic health record by applying static rules or machine learning techniques to the generated feature vectors. For example, the boundary detection module 206 may include or otherwise access the machine learning models and rules database 216. The machine models and rules database 216 includes rule sets and/or classifiers that are configured, e.g., through training, to identify document boundaries and to identify irrelevant portions of text.

For example, the machine models and rules database 216 may include a set of rules that specify that a feature vector representing a handwritten signature indicates the end of a document, or that a feature vector representing a header including one or more of the words “Physician” “Doctor” “Note” or “Summary” indicates the beginning of a document.

As another example, the machine models and rules database 216 may include a first classifier that has been configured through training to receive, as input, feature vectors representing a portion of formatted text and to process the received input to generate, as output, a score indicating a likelihood that the portion of formatted text includes a document boundary or not. For example, the first classifier may be configured to receive vectors representing features of a page of text, e.g., number of lines on page, number of words, diseases or other hotwords mentioned on the page, and to process the vectors to generate a score indicating a likelihood that the portion of formatted text includes a document boundary or not. For example, the first classifier may have learned, through training, that the words “yours sincerely” indicates a document boundary.

As another example, the machine models and rules database 216 may include a second classifier that has been configured through training to receive, as input, feature vectors representing a portion of formatted text and to process the received input to generate, as output, a score indicating a likelihood that the portion of formatted text includes irrelevant text or information. Examples of irrelevant text or information include patient contact information, fax cover sheets, blank pages, pages with junk characters, domain specific non relevant pages such as hospital brochure information, laboratory procedure information.

In some implementations the first classifier and/or the second classifier may include random forests, logistic classifiers, support vector machines, or decision trees.

The boundary detection module 206 uses outputs from the set of rules and machine learning models to segment the formatted text representing the electronic health record into multiple documents corresponding to respective patient encounters with irrelevant portions of text within each document removed. The boundary detection module 206 may provide the multiple documents with irrelevant portions of text removed to the entity extraction and linking module 208.

The entity extraction and linking module 208 is configured to extract medical condition entities and supporting evidence entities referenced in the multiple documents generated by the boundary detection module 206. Example medical condition entities include diseases, disorders or any general medical condition that describes a patient's symptoms, e.g., broken bones or sources of pain. Supporting evidence entities are entities that reference, are linked to or otherwise support medical condition entities. Example supporting evidence entities include but are not limited to medications, administered therapies, symptoms, laboratory results, tests ordered, treatments, assessments, historic medical conditions, the names of medical centers and/or departments thereof visited by the patient, the names of doctors who treated the patient, meals received whilst under the care of said doctor or health center.

The entity extraction and linking module 208 may include a recognition engine component 210 that applies natural language processing techniques or other entity extraction techniques to extract medical condition entities and supporting evidence entities from the multiple documents. In some cases the entity extraction and linking module 208 may receive a list of extracted entities from the recognition engine component 210 and filter the list of extracted entities by removing irrelevant entities, e.g., lexical terms, short terms, context terms, or entities mentioned in reference. In some cases the entity extraction and linking module 208 may further categorize or label extracted entities in the list of extracted entities.

The entity extraction and linking module 208 is further configured to link extracted medical condition entities from a particular document to relevant supporting evidence entities that occur in the same particular document. For example, the entity extraction and linking module 208 may access the medical ontologies database 212 to identify a set of candidate relations between the extracted medical condition entities and any evidence entities that occur in the same document. The entity extraction and linking module 208 may then query the knowledge base system 214 to determine whether any of the relations in the identified set of relations are invalid and to identify any further relations between the extracted medical condition entities and any evidence entities. If invalid relations are identified, the entity extraction and linking module 208 may remove the relations from the candidate set of relations.

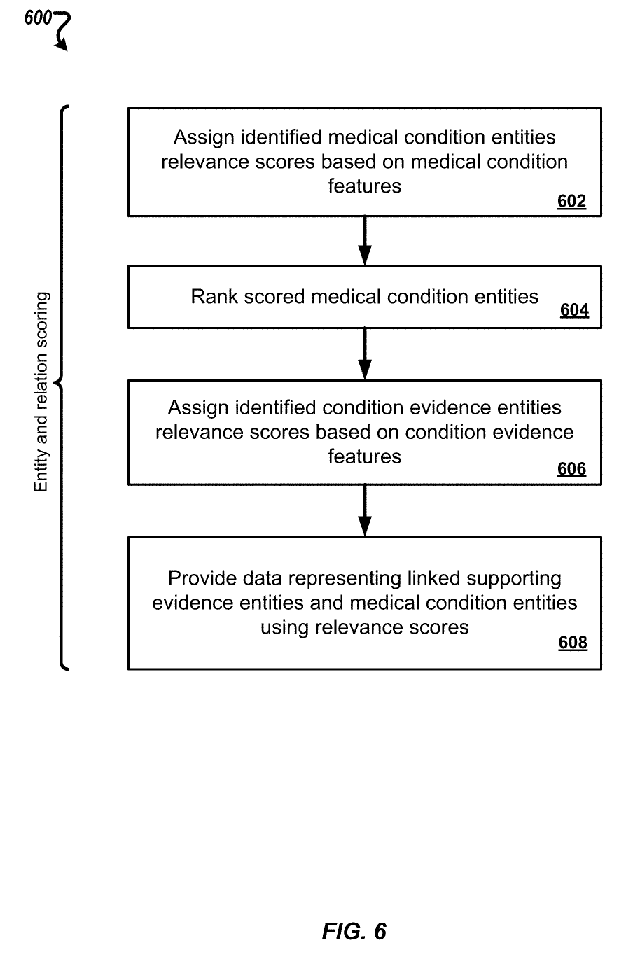

In some implementations, the entity extraction and linking module 208 may score extracted entities and relations between medical condition entities and supporting evidence entities within a same document to determine a most relevant, representative set of medical condition entities and/or relations between medical condition entities and supporting evidence entities. The entity extraction and linking module 208 may score the extracted entities and relations between medical condition entities and supporting evidence entities within a same document based on features of the medical condition entities and supporting evidence entities, as described below with reference to FIG. 6.

Medical ontologies 212 include data representing formal names and definitions of types, properties and interrelationships between entities in a medical domain. For example, medical ontologies 212 may include a compendium of controlled vocabularies in the biomedical sciences, e.g., a unified medical language system (UMLS). For example, the medical ontologies 212 may include a metathesaurus that organizes biomedical information by concept, with each concept having specific attributes defining its meaning and is linked to corresponding concept names in various source vocabularies. The metathesaurus may indicate relationships between concepts, e.g., hierarchical relationships such as disease X “is part of” a class of diseases Y or associative relationships such as condition X “is caused by” behavior Y. The medical ontologies may further include a semantic network that assigns concepts in the metathesaurus one or more semantic types, e.g., organisms, biological functions, chemicals, anatomical structures, that are linked to one another through semantic relationships, e.g., relationships such as “physically related to,” “spatially related to,” “temporarily related to,” “functionally related to” or “conceptually related to.”

Knowledge base system 214 includes a knowledge base that stores structured and unstructured medical information. The knowledge base system 214 may further include an inference engine that can reason about information stored in the knowledge bases and use rules and other forms of logic to deduce new information or highlight inconsistencies. In some implementations the knowledge base system 214 may be configured to receive user input that indicates edits to be made to information stored in the knowledge bases, or edits to made to the rules or forms of logic that are used to deduce new information.

The graphical user interface generator 218 is configured to receive data representing extracted entities and relations between medical condition entities and supporting evidence entities within a same document and to process the received data to generate an interactive graphical user interface (GUI) that visualizes a plain text representation of the electronic health record segmented into multiple documents and provides annotations over the multiple documents that link supporting evidences and medical condition entities. To generate the GUI, the graphical user interface generator 218 may use extracted styling information generated by the data preparation module 204, as described above.

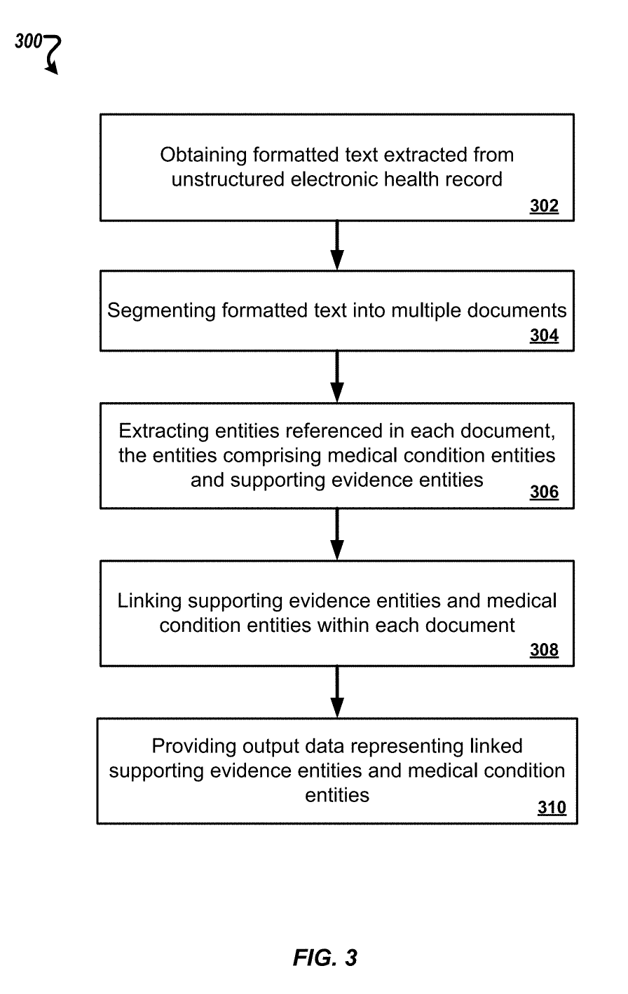

The system 200 may be configured to receive user input through the GUI. For example, a user may view the generated GUI and indicate, through the GUI, edits to the displayed document boundaries or the linked supporting evidences and medical condition entities. For example, a user may select a document boundary and slide the document boundary to a more appropriate place. As another example, a user may remove an annotation that links a supporting evidence entity to a medical condition entity if the link is invalid, or highlight a new supporting evidence entity in an appropriate manner, e.g., colour, to indicate that the new supporting evidence entity should be linked to a corresponding medical condition entity. Generating an interactive GUI based using styling information and data representing extracted entities and relations between medical condition entities and supporting evidence entities within a same document is described in more detail below with reference to FIG. 3.

FIG. 3 is a flowchart of an example process 300 for generating linked medical condition entities and supporting evidence entities from an electronic health record. For convenience, the process 300 will be described as being performed by a system of one or more computers located in one or more locations. For example the system 200 of FIG. 2, appropriately programmed, can perform the process. Although the flowchart depicts the various stages of the process 300 occurring in a particular order, certain stages may in some implementations be performed in parallel or in a different order than what is depicted in the example process 300 of FIG. 3.

The system obtains formatted text extracted from an unstructured electronic health record (step 302). For example, the system may receive input data representing the unstructured electronic health record, e.g., data representing a PDF document. The system may then convert the received input data into a Hypertext Markup Language (HTML) format, e.g., using optical character recognition technology. In some implementations the HTML may preserve the formatting or structure of the original electronic health record, e.g., preserving page breaks, paragraph indentations, headings etc. The system may then extract formatted text by parsing the HTML. In cases where the HTML preserves the page breaks of the original electronic health record, the system may parse the HTML on a page by page basis to generate pages of formatted text that correspond to pages of the original electronic health record.

The system segments the formatted text into multiple documents (step 304). Each document may be associated with a respective document type, e.g., a physician appointment or consultation, laboratory results, admission or discharge notes, letters of referral, procedure notes or a prescription, and a respective document encounter. For example, the segmented formatted text may include multiple documents associated with physician appointments, with each document representing separate physician appointments, e.g., based on a date and time of the appointment. Each of the multiple documents therefore includes a portion or subset of the formatted text, i.e., is smaller than the formatted text obtained with reference to step 302.

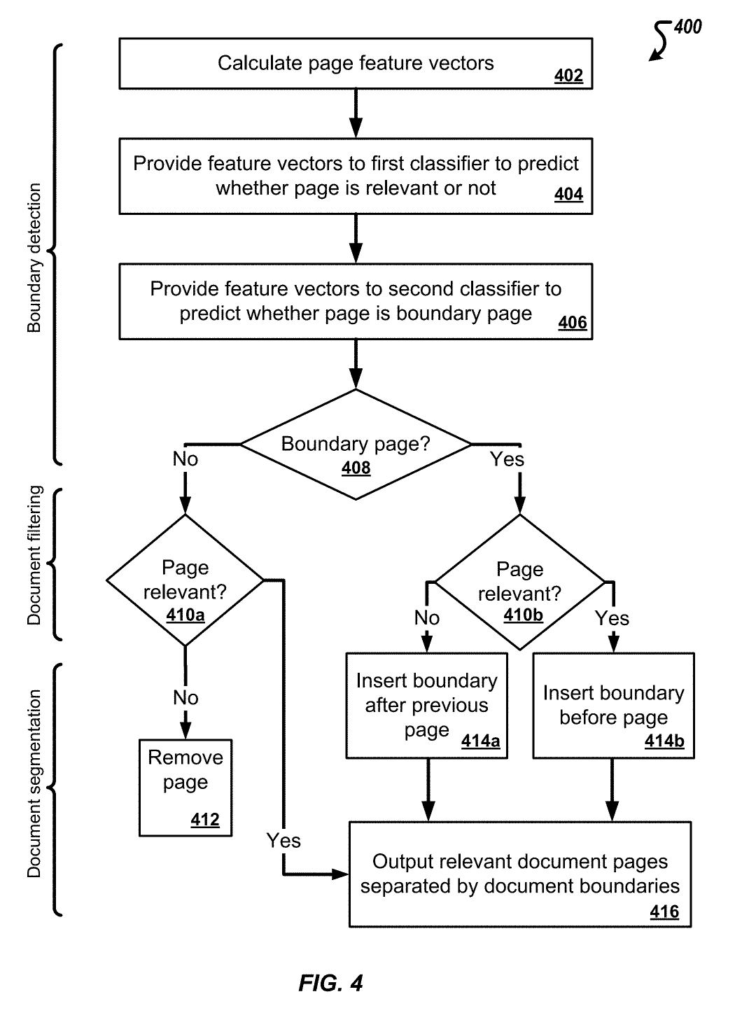

In some implementations segmenting the formatted text into multiple documents may include applying machine learning techniques and/or business rules to automatically segment the formatted text based on the document type and corresponding encounter. Optionally this may further include identifying and removing portions of formatted text that are irrelevant. An example process for applying machine learning techniques to automatically segment formatted text into multiple documents is described below with reference to FIG. 4.

The system extracts, from each of the multiple documents, one or more entities referenced in the document (step 306). The extracted entities include medical condition entities and supporting evidence entities. Example medical condition entities include diseases, disorders or any general medical condition that describes a patient's symptoms, e.g., broken bones or sources of pain. Supporting evidence entities are entities that reference, are linked to or otherwise support medical condition entities. Example supporting evidence entities include but are not limited to medications, administered therapies, symptoms, laboratory results, tests ordered, treatments, assessments, historic medical conditions, the names of medical centers and/or departments thereof visited by the patient, the names of doctors who treated the patient, meals received whilst under the care of said doctor or health center.

In some implementations the system may extract medical condition and supporting evidence entities referenced in each document by applying one or more of natural language processing techniques, entity extraction techniques, or medical ontologies to identify entities of any type that are referenced in each document. For example, the system may include or access a Unified Medical Language System (UMLS) or a clinical Text Analysis and Knowledge Extraction System (cTAKES).

The system may then identify and remove irrelevant entities, e.g., entities that are not medical condition entities or supporting evidence entities. For example, the system may apply domain specific indicators to remove irrelevant entities. Example domain specific indicators include lexical terms, short terms, context terms, or entities mentioned in reference. For example, the system may remove entities that are prepositions or conjunctions, entities that are only one or two characters long such as irrelevant abbreviations, entities mentioned in reference to family members or past medical history, or negated entities, e.g., removing “no” or “denies” before an entity.

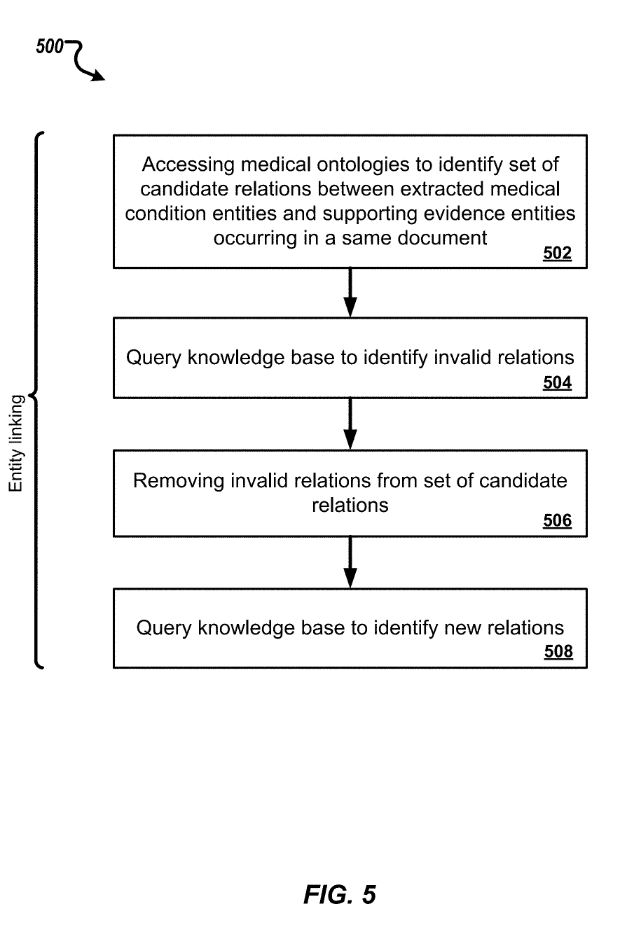

The system links, within each document, one or more of the extracted supporting evidence entities to respective extracted medical condition entities using medical ontologies and/or a medical knowledge base (step 308). For example, the system may query a medical knowledge base or medical ontology with an identified medical condition entity, e.g., a disease. In response the knowledge base may indicate, for example, that a set of medications is typically used to treat the identified medical condition entity, e.g., the disease. The system may then determine whether any of the medications in the set of medications has been identified as a supporting evidence entity in the document. If one or more of the medications in the set of medications has been identified as supporting evidence entities in the document, the system may link the medical condition entity to the supporting evidence entity. An example process for linking extracted medical condition entities to supporting evidence entities is described in more detail below with reference to FIG. 5.

The system provides, for each document, output data representing linked supporting evidence entities and medical condition entities (step 310). In some implementations, as described below with reference to FIG. 6, the system may score linked medical condition entities and supporting evidence entities and provide output data representing a predetermined number of highest scoring linked medical condition and supporting evidence entities, or may provide output data representing linked medical condition and supporting evidence entities whose scores exceed a predetermined threshold.

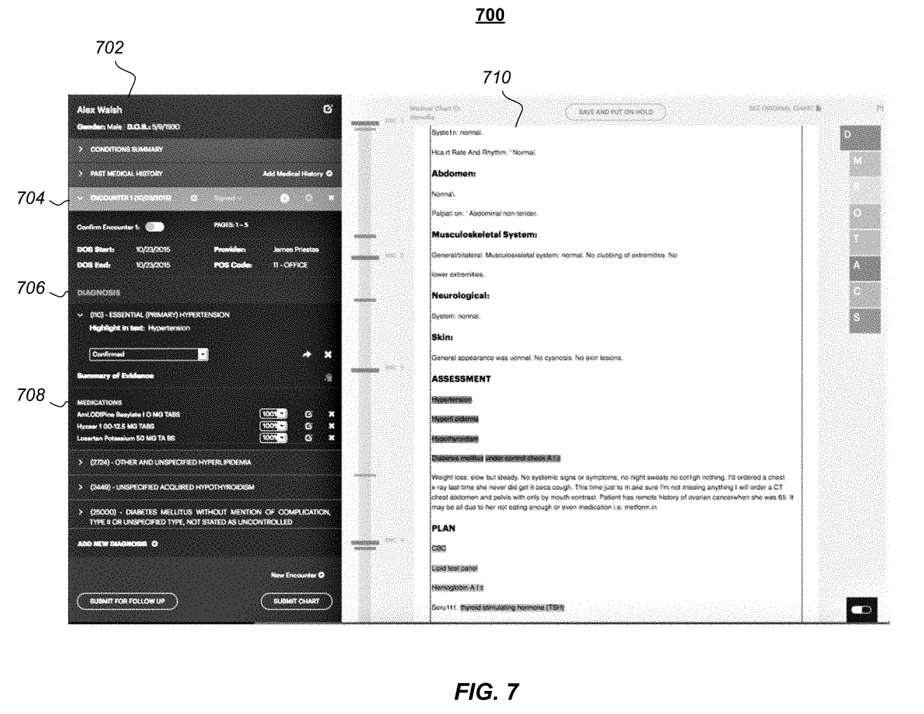

In some implementations, the provided output data may include data representing an interactive graphical user interface (GUI) that displays a visualization of the linked supporting evidences and medical condition entities. The GUI may display the formatted text extracted from the electronic health record, separated into multiple documents, with annotations indicating the linked supporting evidence entities and medical condition entities. For example, the GUI may highlight text representing linked medical condition entities and supporting evidence entities that appear within a same document or throughout all of the multiple documents with a same colour or underline text representing linked medical condition entities and supporting evidence entities. In some cases, e.g., those where the system categorizes identified supporting evidence entities by semantic entity type during step 306 or 308, annotations may indicate categories to which linked medical condition entities and supporting evidence entities belong to, e.g., through a comment or additional marked up text. An example GUI is illustrated below with reference to FIG. 7.

To generate such a GUI, the system may convert the data representing the electronic health record obtained in step 302 into a Hypertext Markup Language format, and parse the converted data to extract electronic health record styling information. Examples of styling information include text headings, text typeface, text colours, or structure of text. The system may use the extracted styling information to generate the interactive graphical user interface, e.g. to generate the display of the formatted text extracted from the electronic health record. By incorporating extracted style information into the GUI, the GUI may be more easily navigated by a user.

In some implementations, the system may apply a continuous learning loop to improve the accuracy of provided output data. For example, the system may further receive user input through the interactive GUI. A user may provide user input through the GUI indicating edits that should be made to the GUI, e.g., edits to the visualized document boundaries (separating the multiple documents) or edits to the linked supporting evidences and medical condition entities. Example edits to the visualized documents boundaries may include moving a document boundary, e.g., in cases where the system has incorrectly separated text into multiple documents as described above with reference to step 304. Example edits to linked supporting evidences and medical condition entities include adding or removing an annotated medical condition entity or supporting evidence entity, e.g., in response to identifying that the system has incorrectly linked a medical condition to a supporting evidence entity or vice versa.

The received user input may be processed and used by the system to update modules or databases included in the system. For example, the received user input may be used to update the knowledge base described above with reference to step 308, e.g., to remove a particular medication from a set of medications that is typically used to treat a particular disease. In this manner, future queries to the knowledge base reflect the user's feedback.

FIG. 4 is a flowchart of an example process 400 for segmenting formatted text extracted from an electronic health record into multiple portions of text. For convenience, the process 400 will be described as being performed by a system of one or more computers located in one or more locations. For example the system 200 of FIG. 2, appropriately programmed, can perform the process. Although the flowchart depicts the various stages of the process 400 occurring in a particular order, certain stages may in some implementations be performed in parallel or in a different order than what is depicted in the example process 400 of FIG. 4.

The system analyzes the formatted text obtained in step 302 of FIG. 3 to calculate multiple feature vectors of numerical features that characterize respective portions of the formatted text (step 402). For example, the system may analyze the formatted text on a page by page basis to determine multiple feature vectors of numerical features that characterize respective pages of the formatted text. The numerical features calculated by the system may be flexible and can be domain specific. Generally, the numerical features may include one or more of lexical features, language features or entity features. Example lexical features include a number of lines, words, nouns or verbs in a portion of formatted text. Example language features include a percentage of words in a domain language such as English, or a number of different languages detected in a portion of text. Example entity features include a number of clinical terms such as diseases, medications, symptoms, tests, names or dates in a portion of text.

The system provides the calculated feature vectors as inputs to a first classifier (step 404). The first classifier is configured to predict whether a portion of text represents a document boundary or not. For example, in some implementations the first classifier may include a rule based system that applies rules to received feature vectors to determine whether the portion of text from which the received feature vectors are taken include a document boundary or not. Alternatively or in addition, the first classifier may include a machine learning model that has been configured through training to predict whether a portion of text represents a document boundary or not. For example, the first classifier may have been trained to process received feature vectors and provide as output a score indicating a likelihood that the portion of text from which the received feature vectors is taken includes a document boundary or not using training feature vectors extracted from pages of multiple electronic health records that are labelled as including a document boundary or not.

As an example, the first classifier may receive feature vectors that indicate that a portion of text includes the words “dosage,” “tablets,” “mg” or “ml”, feature vectors that indicate that the portion of text includes a list of items, and feature vectors that indicate that the portion of text includes a handwritten signature. The first classifier may process said feature vectors using a trained machine learning model to classify the portion of text as a prescription document. The first classifier may then determine that a document boundary is likely to occur directly after the handwritten signature using one or more static rules.

The system provides the calculated feature vectors as inputs to a second classifier, wherein the second classifier has been configured through training to predict whether a portion of text is relevant or not (step 406). For example, the second classifier may have been trained using feature vectors extracted from pages of multiple electronic health records to process received feature vectors and provide as output a score indicating a likelihood that a portion of text from which the received feature vectors is taken from is relevant or not. A portion of text may be considered to be irrelevant if it does not include information relevant to medical condition entities or supporting evidence entities. For example, text representing a patient's contact information may be considered irrelevant, whereas text representing a doctor's contact information may be considered relevant since the address of the doctor may include a reference to the area or department in which the doctor works in, e.g., “Dr. Smith, orthopedic consultant.”

For example, continuing the example above, the second classifier may receive a feature vector that indicates that a portion of text includes a handwritten signature. The second classifier may process the feature vector and determine that the section of text corresponding to the handwritten signature is not relevant.

For each portion of text, the system determines, based on the output from the first classifier, whether the portion of text is a boundary page or not (step 408). In response to determining that a portion of text is not a boundary page, the system determines, based on the output from the second classifier, whether the portion of text is relevant or not (step 410a). In response to determining that the portion of text is not relevant, the system removes the portion of text from the formatted text representations of the electronic health record (step 412). In response to determining that the portion of text is relevant, the system provides the portion of text as output (step 416).

In response to determining that a portion of text is a boundary page, the system determines, based on the output from the second classifier, whether the portion of text is relevant or not (step 410b). In response to determining that the portion of text is not relevant, the system inserts a boundary after the previous portion of text (step 414a). In response to determining that the portion of text is relevant, the system inserts a boundary before the portion of text (step 414b).

The system outputs relevant portions of the formatted text in the form of multiple documents, with each document being separated from other documents by respective document boundaries (step 416).

FIG. 5 is a flowchart of an example process 500 for linking extracted medical condition entities to supporting evidence entities. For convenience, the process 500 will be described as being performed by a system of one or more computers located in one or more locations. For example the system 200 of FIG. 2, appropriately programmed, can perform the process. Although the flowchart depicts the various stages of the process 500 occurring in a particular order, certain stages may in some implementations be performed in parallel or in a different order than what is depicted in the example process 500 of FIG. 5.

The system accesses medical ontologies to identify a set of candidate relations between the extracted medical condition entities and any evidence entities that occur in the same document (step 502). For example, the system may access a Unified Medical Language System (UMLS) that provides a comprehensive thesaurus and ontology of biomedical concepts, and compare the extracted medical condition entities and supporting evidence entities to content in the UMLS to determine whether links exist between the extracted medical condition entities and supporting evidence entities. For example, the UMLS may indicate that a particular disease extracted from one of the multiple documents may be treated by a particular set of therapies and medications. The system may determine whether any of the set of therapies and medications matches the extracted supporting evidences, and, if so, link the matching supporting evidence entities to the medical condition entity.

The system queries a knowledge base to determine whether any of the relations in the identified set of relations are invalid (step 504). For example, as described above with reference to step 310 of FIG. 3, in some implementations the system may apply a continuous learning loop whereby users provide input through an interactive GUI that displays linked medical condition entities and supporting evidence entities as annotations over a representation of the electronic medical record. In these implementations a user may provide feedback indicating that a linked medical condition entity and supporting evidence entity is invalid, i.e., that the medical condition entity should not be linked to the supporting evidence entity. For example, in some implementations a medical condition entity may be erroneously linked to a supporting evidence entity. In response thereto the knowledge base may be updated to indicate that the linked medical condition entity and supporting evidence entity is invalid. As another example, in some implementations a supporting evidence entity may be mentioned in the electronic healthcare record in a different way, e.g., in an alternative spelling, compared to the medical ontology. In this example a user may provide feedback indicating that the supporting evidence entity should be linked to a respective medical condition entity. As another example, in some implementations a user may invalidate a supporting evidence entity that is linked to a medical condition entity in a medical ontology if the link is overly broad and covers all forms of the disease whereas the patient electronic healthcare record refers to a specific variation of the disease where the symptom is not prevalent in the patient.

In response to determining that one or more of the relations are invalid, the system removes the invalid relations from the identified set of relations (step 506).

The system queries the knowledge base to identify new relations between the extracted medical condition entities and any evidence entities that occur in the same document (step 508). As described above with reference to FIGS. 2 and 3, the knowledge graph models domain knowledge and user interactions with the system. The knowledge graph therefore includes valid relations or links between medical condition entities and supporting evidence entities. The system may apply reasoning or inference techniques over the knowledge graph to extract additional or generalize relations between the extracted medical condition entities and supporting evidence entities. For example, in some cases a medical ontology may not be complete, e.g., include edge cases, however a knowledge graph collects domain knowledge from users or other sources via the user reviewing, validating and supplementing the system output, and may therefore be more up to date or include additional relations between the extracted medical condition entities and supporting evidence entities.

FIG. 6 is a flowchart of an example process for scoring linked medical condition entities and supporting evidence entities. For convenience, the process 600 will be described as being performed by a system of one or more computers located in one or more locations. For example the system 200 of FIG. 2, appropriately programmed, can perform the process. Although the flowchart depicts the various stages of the process 600 occurring in a particular order, certain stages may in some implementations be performed in parallel or in a different order than what is depicted in the example process 600 of FIG. 6.

The system assigns the identified medical condition entities a relevance score based on features of the medical condition entities (step 602). Example features of the medical condition entities include features related to the context in which the medical condition entities appear in the document. For example, a medical condition entity that appears in a physician note in a section titled “diagnosis” or “treatment plan” may be assigned a higher relevance score than a medical condition entity that appears in a physician note in a section titled “family medical history.” As another example, a medical condition entity that occurs together with or near to a clinical code within the document may be assigned a higher relevance score than a medical condition entity that does not occur with or near to a clinical code within the document. As another example, a medical condition entity that occurs near other medical condition entities, e.g., as part of a list of medical condition entities, may be assigned a lower relevance score than a medical condition entity that does not occur near other medical condition entities.

Other example features of the medical entities include features relating to the quality of supporting evidence entities linked to the medical condition entities. For example, a medical condition entity that is linked to several supporting evidence entities may be assigned a higher relevance score than a medical condition entity that is linked to none, one or just a few supporting evidence entities. As another example, a medical condition entity that is linked to supporting evidence entities that occur in close proximity to the medical condition entity may be assigned a higher relevance score than a medical condition entity that is linked to supporting evidence entities that do not occur in close proximity to the medical condition entity.

The system ranks the scored medical condition entities to determine a representative subset of condition entities of predetermined size (step 604). For example, the system may determine a representative subset of five top scoring medical condition entities. Alternatively, the system may rank and score the medical condition entities to determine a representative subset of condition entities whose relevance scores exceed a predetermined relevance score threshold, e.g., a subset of condition entities whose relevance scores exceed 80%.

The system assigns the identified supporting evidence entities respective relevance scores based on features of the evidence entities (step 606). The relevance scores may be associated with the relation between the supporting evidence entities and the medical condition entities. For example, a user may assign a score to a medical condition—supporting evidence relation using a review tool output by the system via a GUI. As another example, medical ontologies may include relation scores such as word embeddings similarities of the entity and disease in different texts. As another example relevance scores may be calculated based on the properties of the document such as uniqueness/frequency of the supporting evidence entity in the text or its proximity from an occurrence of the medical condition entity in the text.

The system provides, as output, data representing linked supporting evidence entities and medical condition entities whose relevance scores exceed a predetermined threshold (step 608). For example, the system may filter the identified linked medical condition entities and supporting evidence entities using the relevance scores. In some implementations the system may provide data representing a supporting evidence entity linked to a medical condition entity if one of the supporting evidence entity relevance scores exceeds a predetermined threshold, e.g., if a medical condition entity is assigned a relevance score that exceeds a predetermined relevance threshold, the system may output the medical condition entity and any supporting evidence entities that the medical condition entity is linked to. In some implementations the system may provide data representing a supporting evidence entity linked to a medical condition entity if the combined relevance scores for the medical condition entity and the supporting evidence entity exceed a predetermined threshold. In other implementations the system may rank the linked medical condition entities and supporting condition entities and output data representing a highest scoring number of linked medical condition entities and supporting condition entities, e.g., the top 10 linked medical condition entities and supporting condition entities.

FIG. 7 is an illustration 700 of an example graphical user interface (GUI), as described above with reference to step 310 of FIG. 3. The left panel 802 includes formatted text extracted from an electronic health record. For example, the left panel 802 includes a tab 804 corresponding to a first encounter (an output of the above described document segmentation process). The left panel 802 also includes a diagnosis tab 806 displaying extracted disease entities. The left panel 802 also includes a medications tab 808 for displaying extracted medication entities associated with the diagnosis tab 806.

The right panel 810 shows a plain text with styling representation of a document where extracted medical condition entities and supporting evidences can be validated. The extracted medical condition entities align to text in the document.

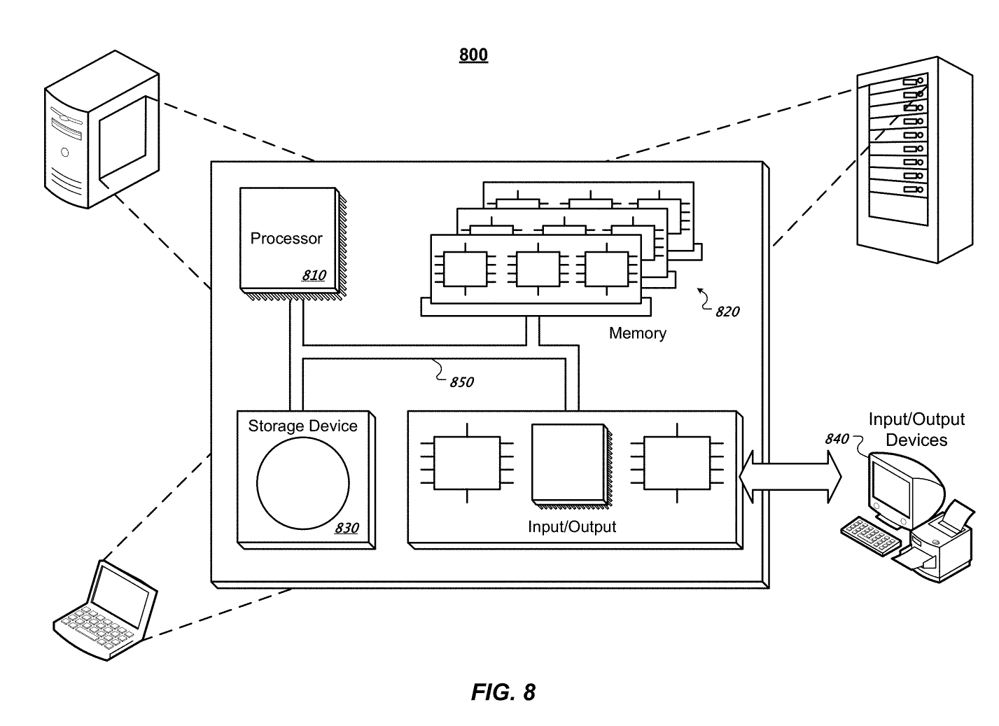

FIG. 8 illustrates a schematic diagram of an exemplary generic computer system 800. The system 800 can be used for the operations described in association with the processes 300-600 described above according to some implementations. The system 800 is intended to represent various forms of digital computers, such as laptops, desktops, workstations, personal digital assistants, servers, blade servers, mainframes, mobile devices and other appropriate computers. The components shown here, their connections and relationships, and their functions, are exemplary only, and do not limit implementations of the inventions described and/or claimed in this document.

The system 800 includes a processor 810, a memory 820, a storage device 830, and an input/output device 840. Each of the components 810, 820, 830, and 820 are interconnected using a system bus 850. The processor 810 may be enabled for processing instructions for execution within the system 800. In one implementation, the processor 810 is a single-threaded processor. In another implementation, the processor 810 is a multi-threaded processor. The processor 810 may be enabled for processing instructions stored in the memory 820 or on the storage device 830 to display graphical information for a user interface on the input/output device 840.

The memory 820 stores information within the system 800. In one implementation, the memory 820 is a computer-readable medium. In one implementation, the memory 820 is a volatile memory unit. In another implementation, the memory 820 is a non-volatile memory unit.

The storage device 830 may be enabled for providing mass storage for the system 800. In one implementation, the storage device 830 is a computer-readable medium. In various different implementations, the storage device 830 may be a floppy disk device, a hard disk device, an optical disk device, or a tape device.

The input/output device 840 provides input/output operations for the system 800. In one implementation, the input/output device 840 includes a keyboard and/or pointing device. In another implementation, the input/output device 840 includes a display unit for displaying graphical user interfaces.

Embodiments and all of the functional operations described in this specification may be implemented in digital electronic circuitry, or in computer software, firmware, or hardware, including the structures disclosed in this specification and their structural equivalents, or in combinations of one or more of them. Embodiments may be implemented as one or more computer program products, i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a stand alone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both.

The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, embodiments may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Embodiments may be implemented in a computing system that includes a back end component, e.g., as a data server, or that includes a middleware component, e.g., an application server, or that includes a front end component, e.g., a client computer having a graphical user interface or a Web browser through which a user may interact with an implementation, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the disclosure or of what may be claimed, but rather as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Thus, particular embodiments have been described. Other embodiments are within the scope of the following claims. For example, the actions recited in the claims may be performed in a different order and still achieve desirable results.

What is claimed is:

1. A computer implemented method for automatically identifying and extracting medical conditions and supporting evidences from electronic health records, the method comprising:
obtaining formatted text extracted from an unstructured electronic health record;
segmenting the formatted text into multiple documents, each document comprising a respective document type and represents a respective document encounter;
extracting, from each document, one or more entities referenced in the document, the entities comprising medical condition entities and supporting evidence entities;
linking, within each document, one or more of the extracted supporting evidence entities to respective extracted medical condition entities using one or more of i) medical ontologies, or ii) a medical knowledge base; and
providing, for each document, output data representing linked supporting evidence entities and medical condition entities.

2. The method of claim 1, wherein segmenting the formatted text into multiple documents comprises:
analyzing the formatted text to calculate multiple feature vectors of numerical features that characterize respective portions of the formatted text;
providing the calculated feature vectors as inputs to a first classifier, wherein the first classifier is configured to predict whether a portion of text represents a document boundary or not; and
segmenting the formatted text into multiple documents by creating document boundaries between portions of text based on outputs received from the first classifier.

3. The method of claim 2, further comprising:
providing the calculated feature vectors as inputs to a second classifier, wherein the second classifier is configured to predict whether a portion of text is relevant or not; and
removing irrelevant portions of text from the formatted text based on outputs received from the second classifier.

4. The method of claim 2, wherein the numerical features comprise one or more of lexical features, language features or entity features.

5. The method of claim 1, wherein evidence entities comprise entities of respective semantic types, the semantic types comprising one or more of i) medications, ii) symptoms, iii) laboratory results, iv) tests ordered, v) treatments, vi) assessments, or vii) historic medical conditions.

6. The method of claim 5, wherein extracting, from each document, one or more entities referenced in the document, wherein the entities comprise condition entities and supporting evidence entities comprises:
applying one or more of i) natural language processing techniques, ii) entity extraction techniques, or iii) medical ontologies to identify one or more medical condition entities and evidence entities in each document; and
identifying and removing irrelevant entities, comprising applying domain specific indicators including one or more of i) lexical terms, ii) short terms, iii) context terms, iv) entities mentioned in reference.

7. The method of claim 6, further comprising categorizing the identified evidence entities by semantic entity type, and wherein the provided data representing linked medical condition entities and supporting evidence entities comprises data indicating which categories the linked medical condition entities and supporting evidence entities belong to.

8. The method of claim 6, wherein linking, within each document, one or more of the extracted supporting evidence entities to respective extracted medical condition entities using one or more of i) medical ontologies, or ii) a medical knowledge base comprises:
accessing medical ontologies to identify a set of candidate relations between the extracted medical condition entities and any evidence entities that occur in the same document;
querying a knowledge base to determine whether any of the relations in the identified set of relations are invalid;
in response to determining that one or more of the relations are invalid, removing the invalid relations from the identified set of relations;
querying the knowledge base to identify new relations between the extracted medical condition entities and any evidence entities that occur in the same document.

9. The method of claim 8, wherein providing, for each document, output data representing linked supporting evidence entities and medical condition entities comprises:
assigning the identified medical condition entities a relevance score based on features of the medical condition, wherein features of the medical condition comprise one or more of i) context within the document, or ii) quality of supporting evidences linked to the medical condition;
ranking the scored medical condition entities to determine a representative subset of condition entities of predetermined size;
assigning the identified supporting evidence entities respective relevance scores based on features of the evidence entities;
providing, as output, data representing linked supporting evidence entities and medical condition entities whose relevance scores exceed a predetermined threshold.

10. The method of claim 9, wherein providing, for each document, output data representing linked supporting evidence entities and medical condition entities comprises providing data representing an interactive graphical user interface that visualizes document boundaries and the linked supporting evidences and medical condition entities as annotations over a plain text representation of the electronic health record.

11. The method of claim 10, wherein providing data representing an interactive graphical user interface that visualizes the linked supporting evidences and medical condition entities as annotations over a plain text representation of the electronic health record comprises:
converting data representing the electronic health record into a Hypertext Markup Language format;
parsing the converted data to extract electronic health record styling information, wherein styling information comprises one or more of i) text headings, ii) text typeface, iii) text colours, iv) structure of text; and
using the extracted styling information to generate the interactive graphical user interface.

12. The method of claim 10, wherein providing, for each document, output data representing linked supporting evidence entities and medical condition entities comprises providing data representing an interactive graphical user interface that visualizes document boundaries and a predetermined number of relevant linked supporting evidences and medical condition entities as annotations over a plain text representation of the electronic health record.

13. The method of claim 10, wherein the plain text representation of the electronic health record comprises relevant portions of text extracted from the electronic health record.

14. The method of claim 10, further comprising:
receiving user input through the interactive graphical user interface, the user input indicating edits to one or more of i) the visualized document boundaries or ii) the linked supporting evidences and medical condition entities; and
updating the knowledge base based on the edits indicated by the received user input.

15. The method of claim 1, further comprising converting unstructured data in the unstructured electronic health record to the formatted text.

16. The method of claim 1, wherein obtaining formatted text extracted from an unstructured electronic health record comprises:
receiving input data representing the unstructured electronic health record;
converting the received input data into a Hypertext Markup Language format; and
extracting formatted text by parsing the Hypertext Markup Language.

17. The method of claim 1 wherein document types comprises one or more of i) doctor appointments, ii) laboratory results, iii) prescriptions, iv) admission or discharge notes, v) letters of referral, or vi) procedure notes.

18. A system comprising:
one or more computers; and
one or more computer-readable media coupled to the one or more computers having instructions stored thereon which, when executed by the one or more computers, cause the one or more computers to perform operations comprising:
obtaining formatted text extracted from an unstructured electronic health record;
segmenting the formatted text into multiple documents, each document comprising a respective document type and represents a respective document encounter;
extracting, from each document, one or more entities referenced in the document, the entities comprising medical condition entities and supporting evidence entities;
linking, within each document, one or more of the extracted supporting evidence entities to respective extracted medical condition entities using one or more of i) medical ontologies, or ii) a medical knowledge base; and
providing, for each document, output data representing linked supporting evidence entities and medical condition entities.

19. The system of claim 18, wherein evidence entities comprise entities of respective semantic types, the semantic types comprising one or more of i) medications, ii) symptoms, iii) laboratory results, iv) tests ordered, v) treatments, vi) assessments, or vii) historic medical conditions.

20. One or more non-transitory computer-readable media having instructions stored thereon that, when executed by one or more processors, cause performance of operations comprising:
obtaining formatted text extracted from an unstructured electronic health record;
segmenting the formatted text into multiple documents, each document comprising a respective document type and represents a respective document encounter;
extracting, from each document, one or more entities referenced in the document, the entities comprising medical condition entities and supporting evidence entities;
linking, within each document, one or more of the extracted supporting evidence entities to respective extracted medical condition entities using one or more of i) medical ontologies, or ii) a medical knowledge base; and
providing, for each document, output data representing linked supporting evidence entities and medical condition entities.
